SEQUENCE LISTING (4 sequences)

SEQ ID NO: 1 - DNA, 21 bp, Artificial Sequence
  Description of Artificial Sequence Synthetic primer
ggaggagctgctgaagatgtg

SEQ ID NO: 2 - DNA, 25 bp, Artificial Sequence
  Description of Artificial Sequence Synthetic primer
cccgagaggtaaagaacaaagactt

SEQ ID NO: 3 - DNA, 17 bp, Artificial Sequence
  Description of Artificial Sequence Synthetic primer
accagtgaagaaagtgt

SEQ ID NO: 4 - DNA, 15 bp, Artificial Sequence
  Description of Artificial Sequence Synthetic primer
cagtgaagcaagtgt

(19) United States
(12) Patent Application Publication
(10) Pub. No.: US 2010/0112589 A1
(43) Pub. Date: May 6, 2010

(54) ALLELE-ALLELE INTERACTIONS OF MTHFR GENE VARIANTS, AND USES THEREOF IN PREDICTING DISEASE RISK

(76) Inventor: Yagang XIE, St. John's (CA)

Correspondence Address: LAHIVE & COCKFIELD, LLP;FLOOR 30, SUITE 3000, ONE POST OFFICE SQUARE, BOSTON, MA, 02109, US

(21) Appl. No.: 12/611,610

(22) Filed: Nov. 3, 2009

Related U.S. Application Data
(60) Provisional application No. 61/110,767, filed on Nov. 3, 2008.

(51) Int. Cl. C12Q 1/68

(52) U.S. Cl. 435/6

(57) ABSTRACT

The invention provides methods of predicting risk of developing a hyper-homocysteine-associated disease in a subject, based on genotyping of the methylenetetrahydrofolate reductase (MTHFR) gene, wherein the risk varies depending on whether the 677T polymorphism and the 1298C polymorphism are present in a cis configuration within a MTHFR gene or not. A preferred hyperhomocysteine-associated disease is myocardial infarction. Kits for predicting risk of developing a hyperhomocysteine-associated disease are also provided.

### RELATED APPLICATION

This application claims priority to U.S. Provisional Application No. 61/110,767, filed Nov. 3, 2008, entitled “Use of Allele-Allele Interaction of MTHFR Gene Variants for Predicting Disease Risk, Prognosis Determination, Molecular Diagnosis, Treatment Guidance and New Drugs Development”, which is specifically incorporated herein by reference in its entirety. The contents of any patents, patent applications, and references cited throughout this specification are hereby incorporated by reference in their entireties.

### SEQUENCE LISTING

The instant application contains a Sequence Listing which has been submitted via EFS-Web and is hereby incorporated by reference in its entirety. Said ASCII copy, created on Nov. 2, 2009, is named NCRI04US.txt, and is 1,292 bytes in size.

### BACKGROUND OF THE INVENTION

The enzyme methylenetetrahydrofolate reductase (MTHFR) catalyzes the NADPH-linked reduction of 5,10-methylenetetrahydrofolate to 5-methylenetetrahydrofolate, a co-substrate for methylation of homocysteine to methionine. Thus, MTHFR is essential for homocysteine metabolism. MTHFR mutations are commonly associated with hyperhomocysteinemia, which has been associated with increased risk of cardiovascular diseases. Two common single nucleotide variants in the coding region of MTHFR gene have been reported to be prevalent in North American, European and Asian populations: a C-to-T change at nucleotide position 677 (677C>T), which results in an Ala222Val mutation in the MTHFR protein, and an A-to-C change at nucleotide position 1298 (1298A>C), which results in a Glu429Ala mutation in the MTHFR protein. Both of these allelic variants have been reported to be associated with elevation in total homocysteine.

The correlation between the risk of cardiovascular diseases, including myocardial infarction (MI), and the 677T allele variant has been extensively investigated by a large number of studies using a variety of different populations and by four recent Meta-analyses. The results of these studies have been conflicting in that a large number of the studies report an association between the 677T allele (especially homozygote of TT) and cardiovascular diseases, whereas no association was seen in others, including a large Meta-analysis. For example, the 677T allele has been reported to be associated with an increased risk for myocardial infarction in a Hungarian population (Kalina, A. and Czeizel, A. E. (2004) Int. J. Cardiol. 97:333-334) and with an increased risk for cardiovascular disease in a Saudi population (Al-Ali, A. K. et al. (2005) Saudi Med. J. 26:1886-1888). Yet in other studies, the 677T allele has been reported to show no association with cardiovascular disease in a Danish population (Frederiksen, J. et al. (2004) Blood 104:3046-3051) and in a Pakistani population (Iqbal, M. P. et al. (2005) J. Mol. Genet. Med. 1:26-32).

Compared with the 677T, the 1298C variant has not been extensively studied and most studies have reported no association with cardiovascular diseases. For example, while one study has reported that the 1298C allele may predispose for early onset coronary artery disease (Szczeklik, A. et al. (2001) Am. J. Med. Genet. 101:36-39), numerous studies have reported no association between the presence of the 1298C variant and the risk of cardiovascular disease (see e.g., Meisel, C. et al. (2001) Atherosclerosis 154:651-658; Rothenbacher, D. et al. (2002) Atherosclerosis 162:193-200; Friso, S. et al. (2002) Clin. Exp. Med. 2:7-12; Haviv, Y. S. et al. (2002) Nephron 92:120-126; Ranjith, N. et al. (2004) Cardiovasc. J. S. Africa 14:127-132; Abu-Amero, K. K. et al. (2003) Arch. Pathol. Lab. Med. 127:1349-1352; Kölling, K. et al. (2004) Am. J. Cardiol. 93:1201-1206).

In view of the foregoing, the relationship between the two MTHFR variants, 677C>T and 1298A>C, and the risk of cardiovascular diseases or other hyperhomocysteine-associated diseases has not been clearly established and continues to be the subject of much debate.

### SUMMARY OF THE INVENTION

This invention provides methods and kits for predicting risk of developing a hyperhomocysteine-associated disease in a subject, based on genotyping of the MTHFR gene. In particular, it has now been discovered that the risk of developing a hyperhomocysteine-associated disease varies depending on whether the 677T polymorphism and the 1298C polymorphism are present in a cis configuration within a MTHFR gene or not. Accordingly, in one aspect, the invention pertains to a method of predicting risk of developing a hyperhomocysteine-associated disease in a subject, the method comprising:
genotyping 677C>T and 1298A>C alleles of a methylenetetrahydrofolate reductase (MTHFR) gene in genomic DNA of the subject;
determining whether a 677T polymorphism is in cis configuration with a 1298C polymorphism; and
predicting risk of developing a hyperhomocysteine-associated disease, wherein:
presence of a 677T polymorphism not in cis configuration with a 1298C polymorphism predicts a reduced risk for development of a hyperhomocysteine-associated disease;
presence of a 1298C polymorphism not in cis configuration with a 677T polymorphism predicts an increased risk for development of a hyperhomocysteine-associated disease; and
presence of a 677T polymorphism in cis configuration with a 1298C polymorphism predicts a significantly increased risk for development of a hyperhomocysteine-associated disease.

In another aspect, the invention pertains to a method of identifying a subject with a reduced risk of developing a hyperhomocysteine-associated disease, the method comprising:

genotyping 677C>T and 1298A>C alleles of a methylenetetrahydrofolate reductase (MTHFR) gene in genomic DNA of the subject;

determining whether a 677T polymorphism is in cis configuration with a 1298C polymorphism; and

identifying a subject as having a reduced risk of developing a hyperhomocysteine-associated disease when a 677T polymorphism is present not in cis configuration with a 1298C polymorphism.

In yet another aspect, the invention pertains to a method of identifying a subject with an increased risk of developing a hyperhomocysteine-associated disease, the method comprising:

genotyping 677C>T and 1298A>C alleles of a methylenetetrahydrofolate reductase (MTHFR) gene in genomic DNA of the subject;

determining whether a 1298C polymorphism is in cis configuration with a 677T polymorphism; and

identifying a subject as having an increased risk of developing a hyperhomocysteine-associated disease when a 1298C polymorphism is present not in cis configuration with a 677T polymorphism.

In yet another aspect, the invention pertains to a method of identifying a subject with a significantly increased risk of developing a hyperhomocysteine-associated disease, the method comprising:

genotyping 677C>T and 1298A>C alleles of a methylenetetrahydrofolate reductase (MTHFR) gene in genomic DNA of the subject;

determining whether a 1298C polymorphism is in cis configuration with a 677T polymorphism; and

identifying a subject as having a significantly increased risk of developing a hyperhomocysteine-associated disease when a 1298C polymorphism is present in a cis configuration with a 677T polymorphism.

In a preferred embodiment, the hyperhomocysteine-associated disease is a cardiovascular disease, such as coronary arterial disease, myocardial infarction or stroke. A particularly preferred cardiovascular disease is myocardial infarction. In other embodiments, the hyperhomocysteine-associated disease can be, for example, thrombosis, an increased risk of a neurotube defect in an offspring of the subject, cancer (e.g., neuroblastoma, colorectal carcinoma), osteoporosis, a neurological disorder (e.g., schizophrenia) or a disorder influenced by folic acid metabolism.

In one embodiment, the subject is a female subject. In another embodiment, the subject is male subject.

In another embodiment, the hyperhomocysteine-associated disease is late onset cardiovascular disease.

In a preferred embodiment, genotyping of the MTHFR gene alleles is performed using polymerase chain reaction (PCR).

In yet another aspect, the invention pertains to a kit for predicting risk of developing a hyperhomocysteine-associated disease in a subject. The kit comprises:

means for genotyping 677C>T and 1298A>C alleles of a methylenetetrahydrofolate reductase (MTHFR) gene in genomic DNA of the subject and for determining whether a 677T polymorphism is in a cis configuration with a 1298C polymorphism; and

instructions for predicting risk of developing a hyperhomocysteine-associated disease based on the genotyping results, wherein the instructions instruct that:

presence of a 677T polymorphism not in a cis configuration with a 1298C polymorphism predicts a reduced risk for development of a hyperhomocysteine-associated disease;

presence of a 1298C polymorphism not in a cis configuration with a 677T polymorphism predicts an increased risk for development of a hyperhomocysteine-associated disease; and

presence of a 677T polymorphism in a cis configuration with a 1298C polymorphism predicts a significantly increased risk for development of a hyperhomocysteine-associated disease.

In a preferred embodiment of the kit, the means for genotyping 677C>T and 1298A>C alleles of a MTHFR gene comprise oligonucleotide primers for amplifying the MTHFR gene. In another preferred embodiment, the means for genotyping 677C>T and 1298A>C alleles of a MTHFR gene further comprise oligonucleotide probes for detection of 677C>T and 1298A>C alleles.

### DETAILED DESCRIPTION OF THE INVENTION

This invention provides methods and kits for predicting risk of developing a hyperhomocysteine-associated disease in a subject. The methods and kits of the invention involve genotyping of the 677C>T and 1298A>C alleles of the methylenetetrahydrofolate reductase (MTHFR) gene in genomic DNA of a subject. The invention is based, at least in part, on the discovery that the risk of developing a hyperhomocysteine-associated disease varies depending on whether the 677T polymorphism and the 1298C polymorphism are present in a cis configuration in a MTHFR gene or not.

In order that the present invention may be more readily understood, certain terms are first defined. Additional definitions are set forth throughout the detailed description.

As used herein, the terms “methylenetetrahydrofolate reductase gene” and “MTHFR gene” each refer to the gene encoding the enzyme that catalyzes the NADPH-linked reduction of 5,10-methylenetetrahydrofolate to 5-methylenetetrahydrofolate. The cDNA encoding human MTHFR, and variants thereof, are described further in PCT Publication Nos. WO 95/33054 and WO 2000/52205.

The term “allele”, as used herein, is intended to refer to one of a group of possible DNA codings (e.g., genes) occupying a particular position (locus) on a chromosome. An individual's “haplotype” is intended to refer to the particular allele that an individual happens to possess at that particular position (locus) on the chromosome, whereas an individual's “genotype” is intended to refer to the set of alleles that the individual happens to possess at that particular position (locus). For example, for diploid organisms such as humans, an individual has two alleles, one on each chromosome, at each position (locus) that make up the individual's genotype, which two alleles may be the same or different on the two chromosomes.

The term “polymorphism”, as used herein, also is intended to refer to one of a group of possible DNA codings (e.g., variant sequences) occupying a particular position (locus) on a chromosome, but the term “polymorphism” is used when the range of possible variant sequences differ by only one or a small number of different nucleotides, whereas the term “allele” is used more broadly, encompassing situations in which the range of possible variant sequences differ more significantly, such as across the length of the protein coding region. For example, a “single nucleotide polymorphism” is intended to refer to one of a group of possible variants at a particular position (locus) on a chromosome, wherein the variants differ only at a single nucleotide position. Thus, single nucleotide polymorphisms of a gene represent different allelic variants of a gene.

The term “677C>T allele” of a MTHFR gene refers to a variant of the MTHFR gene in which there is a C-to-T change at nucleotide position 677. That is, the wild-type MTHFR allele contains a C at nucleotide position 677, whereas the 677C>T variant allele contains a T at nucleotide position 677. The term “677T polymorphism” refers to the variant that contains T at nucleotide position 677.

The term “1298A>C allele” of a MTHFR gene refers to a variant of the MTHFR gene in which there is an A-to-C change at nucleotide position 1298. That is, the wild-type MTHFR allele contains an A at nucleotide position 1298, whereas the 1298A>C variant allele contains a C at nucleotide position 1298. The term “1298C polymorphism” refers to the variant that contains C at nucleotide position 1298.

The term “in cis configuration”, with respect to a variant MTHFR allele, refers to a configuration in which two polymorphisms are present in the same allele of the MTHFR gene. For example, when a 677T polymorphism is present “in a cis configuration” with a 1298C polymorphism, a single MTHFR allele contains both the 677T polymorphism and the 1298C polymorphism such that the encoded MTHFR enzyme contains both the Ala222Val substitution and the Glu429Ala substitution.

The term “hyperhomocysteine-associated disease” refers to a disease or disorder associated with elevated levels of homocysteine. Such elevated levels of homocysteine may be the result of a mutant form of the MTHFR enzyme (due to the presence of a variant MTHFR gene allele), such as a thermolabile form of the enzyme that exhibits decreased enzymatic activity.

The term “a reduced risk for development of a hyperhomocysteine-associated disease”, with respect to the presence of a MTHFR allelic variant, refers to a protective effect provided by the MTHFR allelic variant such that the presence of the MTHFR allelic variant, such as the 677T polymorphic variant, results in less risk of developing a hyperhomocysteine-associated disease than when the wild-type version of the MTHFR allele, such as the 677C wild-type allele, is present.

The term “an increased risk for development of a hyperhomocysteine-associated disease”, with respect to the presence of a MTHFR allelic variant, refers to a disease-promoting effect provided by the MTHFR allelic variant such that the presence of the MTHFR allelic variant, such as the 1298C polymorphic variant, results in a higher risk of developing a hyperhomocysteine-associated disease than when the wild-type version of the MTHFR allele, such as the 1298A wild-type allele, is present. Preferably, the “increased risk” is at least a 1.2 fold increase in risk of developing the disease when the disease-promoting allelic variant is present as compared to when the wild-type allele is present.

The term “a significantly increased risk for development of a hyperhomocysteine-associated disease”, with respect to the presence of two MTHFR allelic variants in cis configuration, refers to disease-promoting effects provided by the MTHFR allelic variants in cis configuration such that the presence of the MTHFR allelic variants in cis configuration, such as the 677T and 1298C polymorphic variants, results in a higher risk of developing a hyperhomocysteine-associated disease than either when the wild-type versions of the MTHFR alleles (such as the 677C and 1298A wild-type alleles), are present or when each of the polymorphic variants is present alone (i.e., not in cis configuration with the other variant). Preferably, the “significantly increased risk” is at least a 2-fold increase, more preferably a 3-fold increase, and even more preferably a 4-fold increase in risk of developing the disease when the disease-promoting allelic variants are present in cis configuration (e.g., 677T and 1298C) as compared to when the wild-type alleles are present.

As used herein, the term “late onset cardiovascular disease” refers to cardiovascular disease (e.g., an incidence of myocardial infarction) that an age of onset that is greater than 50 years of age. In contrast, an “early age of onset” refers to an age of onset that is less than or equal to 50 years of age.

Various aspects of the invention are described in further detail in the following subsections.

### Prediction of Risk of Developing a Hyperhomocysteine-Associated Disease

The invention provides a method of predicting risk of developing a hyperhomocysteine-associated disease in a subject, the method comprising:

genotyping 677C>T and 1298A>C alleles of a methylenetetrahydrofolate reductase (MTHFR) gene in genomic DNA of the subject;

determining whether a 677T polymorphism is in cis configuration with a 1298C polymorphism; and

predicting risk of developing a hyperhomocysteine-associated disease, wherein:
presence of a 677T polymorphism not in cis configuration with a 1298C polymorphism predicts a reduced risk for development of a hyperhomocysteine-associated disease;
presence of a 1298C polymorphism not in cis configuration with a 677T polymorphism predicts an increased risk for development of a hyperhomocysteine-associated disease; and
presence of a 677T polymorphism in cis configuration with a 1298C polymorphism predicts a significantly increased risk for development of a hyperhomocysteine-associated disease.

As described in detail in Example 1, genotypic analysis of the MTHFR gene in a large number of myocardial infarction patients, as compared to normal controls, revealed that the 677T polymorphism has a protective effect in determining disease risk when the 677T polymorphism is not in cis configuration with the 1298C polymorphism. Furthermore, when the 1298C polymorphism is present not in cis configuration with the 677T polymorphism, there is an increased risk of disease (i.e., a 1.2 fold increase in disease risk) as compared to when the wild-type 1298A allele is present. Still further, when the 1298C polymorphism is present in cis configuration with the 677T polymorphism, the protective effect of the 677T polymorphism is no longer observed and, rather, an even greater increased risk of disease (i.e., a 4-fold increase in disease risk) is observed as compared to when the wild type alleles are present.

Accordingly, in another aspect, the invention provides a method of identifying a subject with a reduced risk of developing a hyperhomocysteine-associated disease, the method comprising:

genotyping 677C>T and 1298A>C alleles of a methylenetetrahydrofolate reductase (MTHFR) gene in genomic DNA of the subject;

determining whether a 677T polymorphism is in cis configuration with a 1298C polymorphism; and

identifying a subject as having a reduced risk of developing a hyperhomocysteine-associated disease when a 677T polymorphism is present not in cis configuration with a 1298C polymorphism.

In yet another aspect, the invention provides a method of identifying a subject with an increased risk of developing a hyperhomocysteine-associated disease, the method comprising:

genotyping 677C>T and 1298A>C alleles of a methylenetetrahydrofolate reductase (MTHFR) gene in genomic DNA of the subject;

determining whether a 1298C polymorphism is in cis configuration with a 677T polymorphism; and

identifying a subject as having an increased risk of developing a hyperhomocysteine-associated disease when a 1298C polymorphism is present not in cis configuration with a 677T polymorphism.

In yet another aspect, the invention pertains to a method of identifying a subject with a significantly increased risk of developing a hyperhomocysteine-associated disease, the method comprising:

genotyping 677C>T and 1298A>C alleles of a methylenetetrahydrofolate reductase (MTHFR) gene in genomic DNA of the subject;

determining whether a 1298C polymorphism is in cis configuration with a 677T polymorphism; and

identifying a subject as having a significantly increased risk of developing a hyperhomocysteine-associated disease when a 1298C polymorphism is present in a cis configuration with a 677T polymorphism.

In the methods of the invention for predicting risk of developing a hyperhomocysteine-associated disease in a subject, the 677C>T and 1298A>C alleles of the MTHFR gene are genotyped in genomic DNA of the subject. The genomic DNA can be obtained by standard methods known in the art. For example, genomic DNA can be isolated from peripheral blood using standard salt precipitation methods. The genomic DNA sample may have been previously obtained from the subject and stored for later analysis or, alternatively, the predictive methods of the invention can include an initial step (prior to genotyping) of obtaining a genomic DNA sample from the subject.

In a preferred embodiment, genotyping of the MTHFR gene alleles is performed using polymerase chain reaction (PCR). Various genotyping methods using

PCR are well established in the art, including but not limited to real-time PCR using a Taqman assay (e.g., as described in detail in Example 1) and PCR-restriction fragment length polymorphism (RFLP) assays. Other suitable assays for genotyping include genotyping by mutagenically separated PCR assay (Naghibalhossaini, F. et al. (2008) Clin. Chem. Lab. Med. 46:987-989).

In the methods of the invention, both alleles of the MTHFR gene in the subject are genotyped and it is determined whether the 677T polymorphism is present in a cis configuration with the 1298C polymorphism (as described in more detail in Example 1). In particular, if a subject has any of the following genotypes, then the 677T polymorphism is present in a cis configuration with the 1298C configuration: (i) 677TT/1298CC; (ii) 677TT/1298AC; or (iii) 677TC/1298CC.

A preferred hyperhomocysteine-associated disease is a cardiovascular disease, such as coronary arterial disease, myocardial infarction or stroke. A particularly preferred cardiovascular disease is myocardial infarction.

Preferably, the subject is a human subject. In one embodiment, the subject is a female subject, preferably a female human subject. In another embodiment, the subject is a male subject, preferably a male human subject.

In another embodiment, the hyperhomocysteine-associated disease is late onset cardiovascular disease. As described further in Example 1, the protective effect of the 677T polymorphism (i.e., the reduced risk of developing a hyperhomocysteine-associated disease when the 677T polymorphism is present not in cis configuration with the 1298C polymorphism) and the disease-promoting effect of the 1298C polymorphism (i.e., the increased risk of developing a hyperhomocysteine-associated disease when the 1298C polymorphism is present not in cis configuration with the 677T polymorphism), was more pronounced in subjects with late onset cardiovascular disease. Furthermore, the protective effect of 677T alone and the risk effect of 1298C alone was greater in females than in males, but the significantly increased risk of 677T in cis configuration with 1298C affected both male and female patients.

In other embodiments, the hyperhomocysteine-associated disease can be, for example, thrombosis, an increased risk of a neurotube defect in an offspring of the subject, cancer (e.g., neuroblastoma, colorectal carcinoma), osteoporosis, a neurological disorder (e.g., schizophrenia) or a disorder influenced by folic acid metabolism.

### Kits of the Invention

In another aspect, the invention pertains to kits for carrying out the methods of the invention. For example, in one embodiment, the invention provides a kit for predicting risk of developing a hyperhomocysteine-associated disease in a subject. In one embodiment, the kit comprises:

means for genotyping 677C>T and 1298A>C alleles of a methylenetetrahydrofolate reductase (MTHFR) gene in genomic DNA of the subject and for determining whether a 677T polymorphism is in a cis configuration with a 1298C polymorphism; and

instructions for predicting risk of developing a hyperhomocysteine-associated disease based on the genotyping results, wherein the instructions instruct that:

presence of a 677T polymorphism not in a cis configuration with a 1298C polymorphism predicts a reduced risk for development of a hyperhomocysteine-associated disease;

presence of a 1298C polymorphism not in a cis configuration with a 677T polymorphism predicts an increased risk for development of a hyperhomocysteine-associated disease; and

presence of a 677T polymorphism in a cis configuration with a 1298C polymorphism predicts a significantly increased risk for development of a hyperhomocysteine-associated disease.

Preferably, the means for genotyping 677C>T and 1298A>C alleles of the MTHFR gene comprises a nucleic acid preparation sufficient to detect presence or absence of the allele or polymorphism in a genomic DNA sample from the subject. This nucleic acid preparation includes at least one, and may include more than one, nucleic acid probe or primer, the sequence(s) of which is designed such that the nucleic acid preparation can detect the presence or absence of the allele or polymorphism in a genomic DNA sample from the subject. A preferred nucleic acid preparation includes two or more PCR primers that allow for PCR amplification of a segment of the allele or polymorphic variant. Non-limiting examples of suitable PCR primers for amplification of the alleles of the human MTHFR gene are described in further detail in Example 1. Additionally, suitable probes for detection of the alleles of the human MTHFR gene are described in further detail in Example 1.

Accordingly, in a preferred embodiment of the kit, the means for genotyping 677C>T and 1298A>C alleles of a MTHFR gene comprise oligonucleotide primers for amplifying the MTHFR gene. In another preferred embodiment, the means for genotyping 677C>T and 1298A>C alleles of a MTHFR gene further comprise oligonucleotide probes for detection of 677C>T and 1298A>C alleles.

The means for genotyping the 677C>T and 1298A>C alleles of a MTHFR gene can also include, for example, buffers or other reagents for use in an assay for evaluating the alleles or polymorphisms. The instructions can be, for example, printed instructions for performing the assay for evaluating the alleles or polymorphisms.

As discussed above, once both alleles of the MTHFR gene in the subject are genotyped, the presence of the 677T polymorphism in a cis configuration with the 1298C polymorphism can be determined based on the genotype. In particular, if a subject has any of the following genotypes, then the 677T polymorphism is present in a cis configuration with the 1298C configuration: (i) 677TT/1298CC; (ii) 677TT/1298AC; or (iii) 677TC/1298CC.

Preferably, the kit is designed for use with a human subject, such as a human subject potentially at risk for development of a cardiovascular disease.

The present invention is further illustrated by the following example, which should not be construed as further limiting. The contents of all references, patents and published patent applications cited throughout this application are expressly incorporated herein by reference in their entirety.

### EXAMPLES

### Example 1

### Determination of Disease Risk Using MTHFR Allelic Variants

In this example, a large number of myocardial infarction (MI) patients from a Newfoundland population were genotyped for the 677C>T and 1298A>C alleles of the MTHFR gene, along with normal controls. Furthermore, whether the 677T polymorphism was present in a cis configuration with the 1298C polymorphism was determined. The independent risk for MI associated with each variant was determined, as well as the combined risk for MI associated with both variants together, in particular in the cis configuration.

### Subjects

Blood samples were collected from 1032 consecutive myocardial infarction (MI) patients (640 males and 392 females) and 1014 normal controls (477 males and 537 females) of the genetically isolated Newfoundland population. Patients categorized in the MI group represented those presenting to the emergency department or within one of the Eastern Health's hospitals in St. John's with symptoms and biochemical evidence indicative of MI. Only patients with cardiac Troponin I values greater than 2.0 μg/L (Axsym, Abbott Diagnostics) or greater than 0.5 μg/L (Access II, Beckman-Coulter Corp.) were used in this group. Control subjects were selected from consecutive individuals without prior history of MI or thrombosis presenting to the emergency department for trauma, accidental injury, or other non-cardiac and non-thrombotic related events. Discarded blood samples collected for complete blood count were used for DNA extraction and analysis. Ethics approval for this study was granted by the Human Investigations Committee of Memorial University and by the Health Care Corporation of St. John's.

### Genotyping

Genomic DNA was isolated from peripheral blood using standard salt precipitation methods. Genotyping of the 677C>T and 1298A>C were conducted by using Taq Man SNP genotyping technology on real-time PCR (ABI Prism® 7000 sequence Detection System). The primers and probes for 677C>T were obtained from the Validated TaqMan SNP genotyping kit supplied by Applied Biosystems (ABI; Foster City, Calif.). The primers for 1298A>C were: forward: GGAGGAGCTGCTGAAGATGTG (SEQ ID NO: 1); and reverse: CCCGAGAGGTAAAGAACAAAGACTT (SEQ ID NO: 2). The probes used were: 1298A allele: VIC-ACCAGTGAAGAAAGTGT-TAMRA (SEQ ID NO: 3); 1298C allele: FAM-CAGTGAAGCAAGTGT-TAMRA (SEQ ID NO: 4). PCR reactions were carried out in 96-well optical reaction plates and each reaction consisted of 0.3 μl genomic DNA (100 ng/μl) as template, 2.5 μl of TaqMan Universal PCR Master Mix, 700 nM (each) primer and 200 nM (each) probe in a total volume of 5 μl. After activation of UNG (2 min; 50° C.) and AmpliTaq Gold (10 min; 95° C.), 40 cycles of denaturation (15 sec; 95° C.) and elongation (1 min; 60° C.) were used for two-step PCR. The fluorescent signals of the two reporter dyes were directly determined after PCR. The four distinct clusters were manually categorized as 1298A or 677C (VIC), 1298C or 677T (FAM), 1298A/C or 677C/T (VIC and FAM) and no amplification control (NTC) based on the VIC to FAM ratio.

### Statistical Analysis

The prevalence of each gene variant was calculated by counting the total carrier frequency including heterozygotes and homozygotes. Independent effect of each variant was determined by calculating the sum of prevalence for all possible combined genotypes. The allele frequencies were determined by gene counting. Tests of Hardy Weinberg equilibrium (HWE) were carried out for all loci among MI patients and controls separately by the Chi-square test. Pearson Chi Square statistical analysis was performed using SPSS v10.0 to test the association between genotypes and the prevalence of MI. Odds ratios (OR) were calculated as a measure of the relative risk for MI and were given with 95% CIs. The estimating haplotype frequencies from unphased diploid genotype were calculated via the method of maximum likelihood from genotype data through the use of the expectation-maximization (EM) algorithm under the assumption of HWE. Haplotype frequencies for various marker combinations were estimated for MI patients and controls separately. Linkage disequilibrium between the 677C>T and 1298A>C variants was calculated as D', which ranges from 0 (no linkage disequilibrium) to 1 or −1 (complete linkage disequilibrium).

### Genotyping 677C>T and 1298A>C Variants

To determine the independent risk associated with each variant for MI, both the 677C>T and 1298A>C were genotyped in the entire study population, and the genotype distributions of the two variants were first analyzed separately. The distribution of four possible genotypes for each variant in the MI patients and the controls are given in Table 1. The genotype distributions of both 677C>T (CC, CT and TT) and 1298A>C (AA, AC and CC) in the controls were all in the Hardy-Weinberg equilibrium. The 677T allele (CT and TT) showed a lower prevalence in the MI patients compared with the controls (53.2% vs. 57.30%), (OR=0.847, P=0.062). In contrast, the 1298C allele (AC and CC) presented a significantly increased prevalence in the MI patients compared with the controls (60.37% vs. 52.66%) (OR=1.369, P<0.001). The results of the genotype distribution and haplotype frequency analysis are summarized below in Table 1.

TABLE 1
Genotype distributions and haplotypes frequency estimation in MI
patient and normal control (NC) populations

| | MI | | | |
|---|---|---|---|---|
| Genotype | (n = 1032) | NC (n = 1014) | OR (95% CI) | P value |
| 677 CC | 46.80% | 42.70% | | |
| CT | 43.22% | 45.36% | 0.917 (0.770, 1.091) | 0.328 |
| TT | 9.98% | 11.93% | 0.818 (0.619, 1.081) | 0.157 |
| CT + TT | 53.20% | 57.30% | 0.847 (0.711, 1.009) | 0.062 |
| 1298 AA | 39.63% | 47.34% | | |
| AC | 48.93% | 42.31% | 1.307 (1.098, 1.556) | 0.003 |
| CC | 11.43% | 10.36% | 1.118 (0.846, 1.477) | 0.434 |
| AC + CC | 60.37% | 52.66% | 1.369 (1.149, 1.632) | <0.001 |

### Combined Genotype Analysis

The distribution of combined genotypes between 677C/T and 1298A/C in MI patient and control groups are depicted in Table 2. The genotypes 677CT+TT/1298AA represent all genotypes for which the 677T is not in compound with 1298C. Similarly, the genotypes 677CC/1298AC+CC represent all genotypes where the 1298C is not in compound with 677T. The genotypes 677CT+TT/1298AC+CC represent all genotypes where 677C and 1298T are in compound status (double homozygote, double heterozygote and homozygote heterozygote combination). Finally, the genotypes, 677TT/1298CC+AC and 677CT/1298CC represent all genotypes, except for the double heterozygotes, where the 677T and 1298C are predicted to be in cis configuration (T-C). The prevalence of double homozygosity for both wild type alleles (677CC/1298AA) was found to be very similar in both patient and control groups (11.82% vs. 11.83%). However, there was significant distribution disequilibrium for the combined genotypes, 677CT+TT/1298AA (OR=0.700, P<0.001) and 677CC/1298AC+CC(OR=1.205, P=0.048) in MI patients. Thus, the 677T and 1298C alleles have independent but opposite effects in MI under the circumstances, where the two variants are not in compound status. The significantly reduced prevalence of the combined genotypes 677CT+TT/1298AA indicates a protective effect of 677T allele for MI when it is not compound with the 1298C allele. The significantly increased prevalence of the combined genotype 677CC/1298AC+CC indicates that the MI susceptibility of the 1298C allele is independent.

Examination of all genotypes where the 677T and 1298C alleles occur together (677CT+TT/1298AC+CC) showed an increased prevalence in MI patients (OR=1.221, P=0.056) which did not achieve statistical significance. However, the genotypes of which the 677T and 1298C can be predicted to occur in cis (677TT/1298CC+AC and 677CT/1298CC) showed significantly increased prevalence in MI patients (OR=3.943, P<0.001) compared with the control population. Comparison of odds ratios from 677T only (0.700), 1298C only (1.205) and the T-C in cis (3.943) indicates a co-effect of allele-allele interaction depending on in cis configuration (T-C).

The independent effect of either 677T or 1298C was determined by examining the prevalence of the combined genotypes in cases where the 677T and 1298C are not in compound status. Significant distribution disequilibrium of the combined genotype, 677CC/1298AC+CC(OR: 1.205, P=0.048) and 677CT+TT/1298AA (OR: 0.700, P<0.001) was observed in MI patients compared with the controls. These results indicate independent but opposite roles of MTHFR 677T and 1298C in MI when these variants are not in compound status.

Genetic predisposition in multifactorial disease, such as cardiovascular diseases, results from the co-effect of multiple genes, gene-gene and/or gene-environment interactions. These collectively produce an additive or synergistic effect which affects risk for disease. Individual genetic changes can produce small or insufficient effects to impart significant pathogenic risk. MI is the clinical result of an imbalance in co-effects of interaction among the many risk and protective factors. Complicated intragenic allelic interactions between 677T and 1298C or gene-gene interaction between either MTHFR 677T or MTHFR 1298C with other gene variants could be the cause of discordant results from previous association studies. In the present invention, the 677T and 1298C showed an independent but opposite effect on MI when two of them are not in conjunctive status. The combined genotype 677CT+TT/1298AC+CC represents all of the genotypes where the 677C and 1298T are in compound situation (double homozygote, double heterozygote and homozygote heterozygote combination). Distribution of the combined genotype, 677CT+TT/1298AC+CC, shows a trend toward an increased prevalence in MI patients (OR=1.221, P=0.056) which indicates a possible complex interaction between these two alleles. Intragenic allelic interaction can result from in cis or in trans or both configurations. The combined genotype analysis enables the further investigation of a co-effect of 677T and 1298C when both of them are in cis configuration (677T-1298C). The combined genotypes, 677TT/1298CC+AC and 677CT/1298CC correspond to all but one (double heterozygote) genotypes which the 677T and 1298C are in cis configuration.

Significantly increased prevalence of in cis combined alleles (677T-1298C) in MI patients (OR=3.943, P<0.001) compared with the control population strongly indicates a co-effect via allele-allele interaction pending on in cis configuration. In this allele-allele interaction, the protective effect of 677T seems to be off-set by 1298C when both of them are in cis. Furthermore, the risk effect of 1298C was shown to be greatly enhanced by the in cis 677T allele compared with the situation that the 1298C is in non-conjunction with 677T (OR; 3.943 vs 1.205). As distribution disequilibrium of both 677T and 1298C was only detected in later onset patients, it appears that the protective effect of 677T and risk effect of 1298C are both weak for MI. The protective effect of 677T in MI condition is less gender influenced. Considering the risk effect of 1298C alone for MI is greater in females indicates that the effect of 1298C alone is very weak and can be influenced by other genetic (e.g. female hormones) or environment factor (e.g. contraception medications). However, the enhanced risk effect from in cis 677T-1298C_(677TT/1298CC+AC and 677CT/1298CC) affects both male (OR=2.926, P=0.021) and female patients (OR=5.670, P=0.001) with greatly augmented odds ratios compared with those obtained in the 1298C alone genotype in both male (2.926 vs 1.142) and female patients (5.670 vs 1.634).

### Calculation of Estimated Haplotype Frequency

To confirm the in cis interaction between the 677T and 1298C, all possible combined genotypes between the 667C/T and 1298A/C were calculated at the haplotype level. The calculated haplotype frequencies for all four possible genotypes in both MI and control populations are also presented in the Table 2. The 677T-1298A haplotype shows significantly reduced frequency in the MI patients compared with the controls (OR: 0.782; P<0.001). In contrast, the 677T-1298C haplotype had a significantly increased frequency in the MI patients compared with the controls (OR: 3.206; P<0.001).

Utilization of haplotypes in association studies for the commonly occurring variants can increase power over single-allele studies. Therefore, the estimated haplotype frequencies for all four possible combined genotypes were calculated. The haplotype of 677T-1298A showed a significantly reduced frequency in MI patients compared to the controls, which indicates a protective role of MTHFR 677T only when position 1298 is wild type (A). Furthermore, a significantly increased frequency of the haplotype of 677T-1298C was observed in the MI patients compared with the controls and shows a similar odds ratio with the ones observed in analysis of combined genotype which represent in cis combined alleles (677T-1298C).

All of these data from combined genotype analysis and haplotype frequency calculation indicate an interaction between 677T and 1298C depending on the in cis or in trans configuration of these two alleles. Both of these variants code for an amino acid substitution that when occurring together represent changes in amino acids 177 residues apart. Therefore, the co-effect from in cis interaction between 677T and 1298C can attribute to a change in conformation of MTHFR protein due to the two missense changes in the same peptide. This complicated allele-allele interaction between 677T and 1298C presents an explanation for the highly conflicting results of the large number of previous studies.

TABLE 2
Distributions of combined genotype and haplotypes frequency estimation
in MI patient and normal control (NC) populations

| 677C/T | 1298A/C | MI (n = 1032) | NC (n = 1014) | OR (95% CI) | P value |
|---|---|---|---|---|---|
| CC | AA | 122 (11.82%) | 120 (11.83%) | 0.999 (0.764, 1.306) | |
| | AC | 260 (25.19%) | 212 (20.91%) | 1.274 (1.036-1.566) | 0.024 |
| | CC | 101 (9.79%) | 101 (9.96%) | 0.981 (0.733-1.311) | 0.941 |
| CT | AA | 209 (20.25%) | 249 (24.56%) | 0.780 (0.633-0.961) | 0.02 |
| | AC | 223 (21.61%) | 211 (20.81%) | 1.049 (0.849-1.297) | 0.666 |
| | CC | 14 (1.36%) | 0 (0%) | | |
| TT | AA | 78 (7.56%) | 111 (10.95%) | 0.665 (0.491-0.901) | 0.009 |
| | AC | 22 (2.13%) | 6 (0.95%) | 3.659 (1.478-9.063) | 0.004 |
| | CC | 3 (0.29%) | 4 (0.39%) | 0.736 (0.164-3.297) | 0.724 |

677T only

| | | | | |
|---|---|---|---|---|
| 677CT + TT/1298AA | 287 (27.81%) | 360 (35.51%) | 0.700 (0.580, 0.844) | <0.001 |

1298C only

| | | | | |
|---|---|---|---|---|
| 677CC/1298AC + CC | 361 (34.98%) | 313 (30.87%) | 1.205 (1.002, 1.449) | 0.048 |

Genotypes of all T/C in compound status

| | | | | |
|---|---|---|---|---|
| 677CT + TT/1298AC + CC | 262 (25.39%) | 221 (21.80%) | 1.221 (0.995, 1.498) | 0.056 |

Predictable genotypes of T-C in cis configuration

| | | | | |
|---|---|---|---|---|
| 677TT/1298CC + AC | 25 (2.42%) | 10 (0.98%) | | |
| 677CT/1298CC | 14 (1.36%) | 0 | | |
| Total | 39 (3.78%) | 10 (0.98%) | 3.943 (1.958, 7.943) | <0.001 |

Haplotype frequencies

| | | | | |
|---|---|---|---|---|
| 677C-1298A | 35.74% | 34.93% | 1.038 (0.913, 1.180) | 0.572 |
| 677C-1298C | 32.67% | 30.46% | 1.106 (0.970, 1.262) | 0.133 |
| 677T-1298A | 28.36% | 33.58% | 0.782 (0.685, 0.894) | <0.001 |
| 677T-1298C | 3.23% | 1.05% | 3.206 (1.956, 5.255) | <0.001 |

### Influence of Gender and Onset Age

The distributions of the two MTHFR variants were further analyzed by sub-grouping MI patients and controls according to age and sex. The results are summarized in Table 3. In the age based population study, the two variants were analyzed in MI patients divided into two groups consisting of those with an early age of onset (<50 years) and those with a later age of onset (>50 years). The control population was also divided into the two corresponding age groups. Neither the 677T nor the 1298C alleles showed any significant distribution disequilibrium in early onset MI patients. The 677T alone genotypes (677CT+TT/1298AA) showed significantly reduced prevalence in the later onset MI patient group (OR: 0.622, P<0.001) compared with the aged matched controls. The 677T-1298C in cis genotypes (677TT/1298CC+AC and 677CT/1298CC) had a significantly higher prevalence in the later onset MI patient group (OR: 4.467, P=0.001) compared with the age matched controls. In the gender based subpopulation study, the prevalence of 677T alone genotypes (677CT+TT/1298AA) was significantly reduced in male patients with MI (OR=0.643, P=0.001), and tended to be lower in female patients (OR=0.764, P=0.064). The prevalence of 1298C alone genotypes (677CC/1298CA+CC) was significantly higher in female patients (OR=1.634, P<0.001). However, the 677T-1298C in cis genotypes (677TT/1298CC+AC and 677CT/1298CC) was significantly associated with MI in both male (OR=2.926, P=0.021) and female patients (OR=5.670, P=0.001) and the odds ratios were much higher than with genotypes where the 1298C is present without any 677T allele in both male (2.926 vs 1.142) and female patients (5.670 vs 1.634).

As distribution disequilibrium of both 677T and 1298C was only detected in later onset patients, the protective effect of 677T and risk effect of 1298C are both weak for MI. The protective effect of 677T in MI condition is less gender influenced. Considering the risk effect of 1298C alone for MI is greater in females indicates that the effect of 1298C alone is very weak and can be influenced by other genetic (e.g., female hormones) or environment factors (e.g., contraception medications). However, the enhanced risk effect from in cis 677T-1298C_(677TT/1298CC+AC and 677CT/1298CC) affects both male (OR=2.926, P=0.021) and female patients (OR=5.670, P=0.001) with greatly augmented odds ratios compared with those obtained in the 1298C alone genotype in both male (2.926 vs 1.142) and female patients (5.670 vs 1.634).

TABLE 3
Distribution of MTHFR genotypes among MI patients with different onset
age compared with age and gender matched normal controls (NC). (based on the
subgroup.)

| | MI | NC | OR (95% CI) | P value |
|---|---|---|---|---|
| Age ≦ 50 | 139 | 608 | | |
| 677 CT + TT/1298AA | 43 (30.94%) | 207 (34.05%) | 0.868 (0.583, 1.290) | 0.550 |
| 677CC/1298AC + CC | 51 (36.69%) | 186 (30.59%) | 1.315 (0.894, 1.934) | 0.189 |
| Known in cis (T-C) | 1 (0.72%) | 7 (1.15%) | 0.622 (0.076, 5.098) | 1.000 |
| Age > 50 | 893 | 406 | | |
| 677 CT + TT/1298AA | 244 (27.32%) | 153 (37.68%) | 0.622 (0.485, 0.797) | <0.001 |
| 677CC/1298AC + CC | 310 (34.71%) | 127 (31.28%) | 1.168 (0.909, 1.501) | 0.229 |
| Known in cis (T-C) | 38 (4.26%) | 4 (0.99%) | 4.467 (1.583, 12.600) | 0.001 |
| Males | 640 | 477 | | |
| 677 CT + TT/1298AA | 176 (27.50%) | 177 (37.11%) | 0.643 (0.499, 0.829) | 0.001 |
| 677CC/1298AC + CC | 220 (34.38%) | 150 (31.45%) | 1.142 (0.887, 1.471) | 0.335 |
| Known in cis (T-C) | 23 (3.59%) | 6 (1.26%) | 2.926 (1.182, 7.244) | 0.021 |
| Females | 392 | 537 | | |
| 677 CT + TT/1298AA | 111 (28.32%) | 183 (34.08%) | 0.764 (0.576, 1.014) | 0.064 |
| 677CC/1298AC + CC | 141 (35.97%) | 163 (30.35%) | 1.634 (1.244, 2.145) | <0.001 |
| Known in cis (T-C) | 16 (4.08%) | 4 (0.74%) | 5.670 (1.881, 17.095) | 0.001 |

What is claimed is:

1. A method of predicting risk of developing a hyperhomocysteine-associated disease in a subject, the method comprising:
a) genotyping 677C>T and 1298A>C alleles of a methylenetetrahydrofolate reductase (MTHFR) gene in genomic DNA of the subject;
b) determining whether a 677T polymorphism is in cis configuration with a 1298C polymorphism; and
c) predicting risk of developing a hyperhomocysteine-associated disease, wherein:
i. presence of a 677T polymorphism not in a cis configuration with a 1298C polymorphism predicts a reduced risk for development of a hyperhomocysteine-associated disease;
ii. presence of a 1298C polymorphism not in a cis configuration with a 677T polymorphism predicts an increased risk for development of a hyperhomocysteine-associated disease; and
iii. presence of a 677T polymorphism in a cis configuration with a1298C polymorphism predicts a significantly increased risk for development of a hyperhomocysteine-associated disease.

2. The method of claim 1, wherein the hyperhomocysteine-associated disease is a cardiovascular disease.

3. The method of claim 2, wherein the cardiovascular disease is coronary arterial disease, myocardial infarction or stroke.

4. The method of claim 2, wherein the cardiovascular disease is myocardial infarction.

5. The method of claim 1, wherein the hyperhomocysteine-associated disease is selected from the group consisting of thrombosis, an increased risk of a neurotube defect in an offspring of the subject, cancer, osteoporosis, neurological disorders and disorders influenced by folic acid metabolism.

6. The method of claim 1, wherein the subject is a female subject.

7. The method of claim 1, wherein the subject is male subject.

8. The method of claim 2, wherein the cardiovascular disease is a late onset cardiovascular disease.

9. The method of claim 1, wherein genotyping is performed using polymerase chain reaction (PCR).

10. A method of identifying a subject with a reduced risk of developing a hyperhomocysteine-associated disease, the method comprising:
a) genotyping 677C>T and 1298A>C alleles of a methylenetetrahydrofolate reductase (MTHFR) gene in genomic DNA of the subject;
b) determining whether a 677T polymorphism is in cis configuration with a 1298C polymorphism; and
c) identifying a subject as having a reduced risk of developing a hyperhomocysteine-associated disease when a 677T polymorphism is present not in a cis configuration with a 1298C polymorphism.

11. The method of claim 10, wherein the hyperhomocysteine-associated disease is a cardiovascular disease.

12. The method of claim 11, wherein the cardiovascular disease is coronary arterial disease, myocardial infarction or stroke.

13. The method of claim 11, wherein the cardiovascular disease is myocardial infarction.

14. The method of claim 10, wherein the hyperhomocysteine-associated disease is selected from the group consisting of thrombosis, an increased risk of a neurotube defect in an offspring of the subject, cancer, osteoporosis, neurological disorders and disorders influenced by folic acid metabolism.

15. The method of claim 10, wherein the subject is a female subject.

16. The method of claim 10, wherein the subject is male subject.

17. The method of claim 11, wherein the cardiovascular disease is a late onset cardiovascular disease.

18. The method of claim 10, wherein genotyping is performed using polymerase chain reaction (PCR).

19. A method of identifying a subject with an increased risk of developing a hyperhomocysteine-associated disease, the method comprising:
a) genotyping 1298A>C and 677C>T alleles of a methylenetetrahydrofolate reductase (MTHFR) gene in genomic DNA of the subject;
b) determining whether a 1298C polymorphism is in cis configuration with a 677T polymorphism; and
c) identifying a subject as having an increased risk of developing a hyperhomocysteine-associated disease when a 1298C allele is present not in a cis configuration with a 677T polymorphism.

20. The method of claim 19, wherein the hyperhomocysteine-associated disease is a cardiovascular disease.

21. The method of claim 20, wherein the cardiovascular disease is coronary arterial disease, myocardial infarction or stroke.

22. The method of claim 20, wherein the cardiovascular disease is myocardial infarction.

23. The method of claim 19, wherein the hyperhomocysteine-associated disease is selected from the group consisting of thrombosis, an increased risk of a neurotube defect in an offspring of the subject, cancer, osteoporosis, neurological disorders and disorders influenced by folic acid metabolism.

24. The method of claim 19, wherein genotyping is performed using polymerase chain reaction (PCR).

25. A method of identifying a subject with a significantly increased risk of developing a hyperhomocysteine-associated disease, the method comprising:
a) genotyping 677C>T and 1298A>C alleles of a methylenetetrahydrofolate reductase (MTHFR) gene in genomic DNA of the subject;
b) determining whether a 677T polymorphism is in cis configuration with a 1298C polymorphism; and
c) identifying a subject as having a significantly increased risk of developing a hyperhomocysteine-associated disease when a 677T polymorphism is present in a cis configuration with a 1298c polymorphism.

26. The method of claim 25, wherein the hyperhomocysteine-associated disease is a cardiovascular disease.

27. The method of claim 26, wherein the cardiovascular disease is coronary arterial disease, myocardial infarction or stroke.

28. The method of claim 26, wherein the cardiovascular disease is myocardial infarction.

29. The method of claim 25, wherein the hyperhomocysteine-associated disease is selected from the group consisting of thrombosis, an increased risk of a neurotube defect in an offspring of the subject, cancer, osteoporosis, neurological disorders and disorders influenced by folic acid metabolism.

30. The method of claim 25, wherein the subject is a female subject.

31. The method of claim 25, wherein the subject is male subject.

32. The method of claim 26, wherein the cardiovascular disease is a late onset cardiovascular disease.

33. The method of claim 25, wherein genotyping is performed using polymerase chain reaction (PCR).

34. A kit for predicting risk of developing a hyperhomocysteine-associated disease in a subject, the kit comprising:
a) means for genotyping 677C>T and 1298A>C alleles of a methylenetetrahydrofolate reductase (MTHFR) gene in genomic DNA of the subject and for determining whether a 677T polymorphism is in a cis configuration with a 1298C polymorphism; and
b) instructions for predicting risk of developing a hyperhomocysteine-associated disease, wherein the instructions instruct that:
i. presence of a 677T polymorphism not in a cis configuration with a 1298C polymorphism predicts a reduced risk for development of a hyperhomocysteine-associated disease;
ii. presence of a 1298C polymorphism not in a cis configuration with a 677T polymorphism predicts an increased risk for development of a hyperhomocysteine-associated disease; and
iii. presence of a 677T polymorphism in a cis configuration with a 1298C polymorphism predicts a significantly increased risk for development of a hyperhomocysteine-associated disease.

35. The kit of claim 34, wherein the means for genotyping 677C>T and 1298A>C alleles of a MTHFR gene comprise oligonucleotide primers for amplifying the MTHFR gene.

36. The kit of claim 35, wherein the means for genotyping 677C>T and 1298A>C alleles of a MTHFR gene further comprise oligonucleotide probes for detection of 677C>T and 1298A>C alleles.
